(19) United States
(12) Patent Application Publication
(10) Pub. No.: US 2010/0241100 A1
(43) Pub. Date: Sep. 23, 2010

(54) Real-time multimode neurobiophysiology probe

(76) Inventors: Walter Blumenfeld, Airville, PA (US); Jehuda Peter Sepkuty, Redmond, WA (US); Gregory Solyar, Baltimore, MD (US)

Correspondence Address: NADEJDA REINGAND, 7 CLIFTON CT., BALTIMORE, MD, 21208, US

(21) Appl. No.: 12/381,999

(22) Filed: Mar. 19, 2009

(51) Int. Cl. A61M 37/00; A61B 5/00

(52) U.S. Cl. 604/503; 600/584; 600/573; 604/66

(57) ABSTRACT

Apparatus and methods in which very small volumes of material may be extracted, delivered, interrogated or stimulated via optical, electromagnetic or mechanical means, in vivo or in vitro, for site-specific detection, characterization, stimulation, diagnostics or therapy, comprising optical, fluidic, chemical, electromagnetic and biological techniques applied via a microprobe in a single intra-parenchymal tissue perforation procedure in the brain. The primary use of the device is in neuroscience research, clinical diagnostics and therapeutics applications in the brain, however, the device may also be beneficially applied to other organs and biological systems. Human clinical applications may include neurosurgical intra-operative monitoring, extra-operative chronic monitoring of devices introduced in an operation, and diagnostic monitoring combined with simultaneous neuroimaging.

### BACKGROUND

1. Field of the Invention

This invention relates generally to the field of neuroscience, biotechnology and medical instrumentation, and particularly to molecular sampling, delivery and characterization methods applied in conjunction with optical, electromagnetic or electrochemical interrogation or excitation by means of a minimally-invasive probe at a designated site in the brain.

2. Related Art

U.S. Pat. No. 6,584,335 to Hans-Peter Haar describes an end-sealed hollow needle having a permeable area allowing size-limited fluid-borne molecules to be coupled via evanescent field effects through a semi-permeable coating to an optical fiber or waveguide positioned in the needle cavity. This allows optical interrogation by quantum-cascadelaser-excited multiple-wavelength attenuated total reflectance spectroscopy (ATR) in the 7 to 13-micron wavelength region. This enables detection and quantification of blood glucose concentration, which, in principle, might be used to control the administration of insulin through the interior of the hollow needle surrounding the optical fiber. The efficacy of this device is dependent on unobstructed function of the permeable area of the hollow needle and on the stability of the evanescent-field coupling efficiency of the semipermeable coating of the optical fiber or light-guide; this is subject to variability with temperature and requires probe temperature measurement and heating control in order to maintain function. Another confounding effect on the ATR analysis is the possibility of fouling the semi-permeable membrane with a local concentration of small molecules or an adherent fluid-borne substance, thereby aliasing the spectral data.

US2007/0142714A1 to Daniel L. Shumate describes a needle containing bundled microtubes and optical sensing fibers. Therapeutic fluids may be delivered and extracted through microtubes by pulsatile micro-pumps. Temperature, pH and PO2 may be measured by separate fibers, which may or may not have chemical-sensing or temperature-sensing coatings. This device has no means of sample particulate or molecular size selectivity, and no means for concentration or amplification of the desired analyte(s). Target applications include tumor diagnostics, orthopedic joint and back surgery, and opthalmic surgery. Opthalmic probes are also referenced in U.S. Pat. No. 5,643,250 and U.S. Pat. No. 6,520,955.

### SUMMARY OF THE INVENTION

The invention is a a multimodal probe for applications in neuroscience research and clinical diagnostics. Intended for use in a single intra-parenchymal perforation procedure in the brain, the device provides a minimally invasive site-specific means for:

Sampling and delivery of picoliter/microliter fluid volumes, with selective size control of suspended material or molecules. This includes delivery of chemical and/or biological factors or a physical response in a closed loop fashion: sampling-analysis-delivery.

Optical interrogation of internal fluid or external tissue or fluid for analysis or differentiation by spectroscopic properties of the sample or of chemical or biological sensor materials exposed to the sample.

Directed optical excitation or stimulation of tissue or fluid for diagnostic or therapeutic purposes, including optical activation of chemical or biological agents.

Electromagnetic or electrochemical sensing or excitation of tissue or fluid for diagnostic or therapeutic procedures.

Monitoring of physical conditions including intracranial pressure and probe temperature.

Combinations of two or more of these techniques, applied simultaneously or sequentially at the same site, or at multiple sites along the axis of a single probe, or at multiple sites with multiple probes.

### BRIEF DESCRIPTION OF THE DRAWINGS

FIG. 1 depicts a first embodiment of the invention for monitoring Oxygen concentration, EEG and intracranial pressure, while extracting fluid-borne particulates and molecules of less than 5 microns diameter from interstitial fluid in the brain.

FIG. 2 shows a simpler second embodiment of the invention, in which the optical fiber is fixed and terminated at the probe manifold, allowing unobstructed fluid extraction and delivery.

FIG. 3 shows a third embodiment of the invention, in which the optical fiber may be alternately extended or retracted.

FIG. 4 shows a fourth embodiment of the invention, in which the internal probe volume is minimized to provide reduced phase lag in fluid sampling and delivery.

FIG. 5 shows a fifth embodiment of the invention, in which an additional optical source may provide direct optical stimulation of tissue, or photolysis of an inert reagent to release a biologically active breakdown product.

FIG. 6 shows a sixth embodiment of the invention, in which the probe is configured for fibrinolytic “clot-busting” stroke therapy with monitored tPA delivery.

FIG. 7 shows a seventh embodiment of the invention, configured for electrical deep brain stimulation in conjunction with optical interrogation and fluid irrigation, delivery or sample extraction.

FIG. 8 shows an eighth embodiment of the invention, in which the device is configured as a patch clamp microprobe for electronic ion channel measurements enhanced by simultaneous optical spectroscopic interrogation.

### DESCRIPTION OF THE INVENTION

### Device Structure

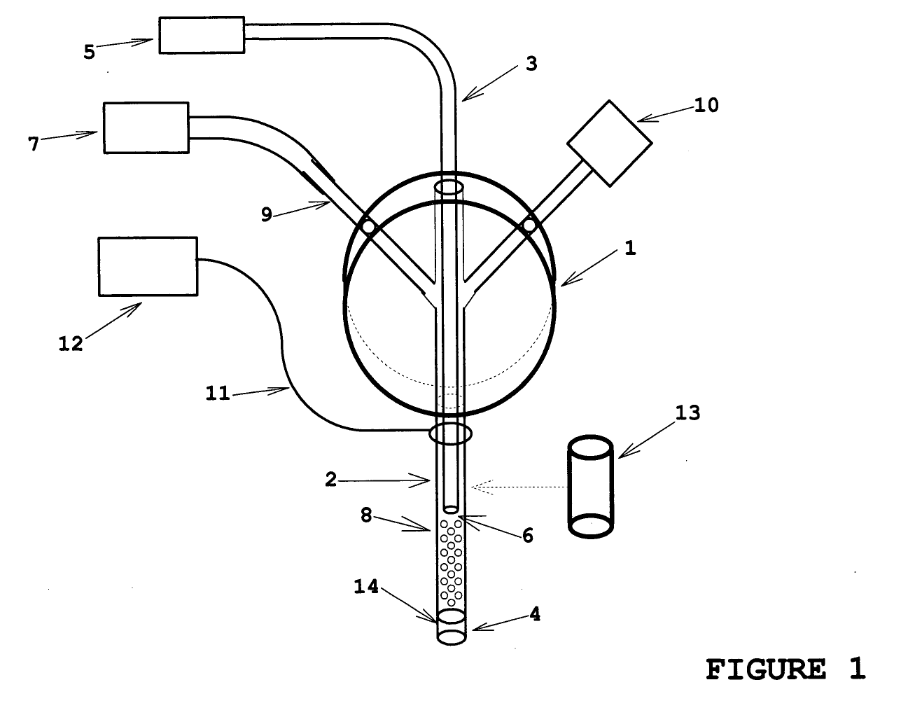

The device structure shown in FIG. 1 comprises multiple sections of tubing, retained in intersecting bores in a multi-port manifold body 1. The manifold bores provide access for fluid extraction or delivery, or serve as conduits of means for interrogation or excitation of fluid or tissue via optical, electrical, chemical, magnetic or mechanical sensors or transducers. Some of these means may reside coaxially within one or more of the intersecting bores. The manifold body 1 may be fabricated from stainless steel, titanium, ceramic, glass, acetyl (or some other polymer). The tubing must also be a biocompatible material, not necessarily the same as that of the manifold body. Appropriate material selection allows fabrication of probes which are compatible with MRI or other imaging procedures.

### Preferred Embodiments

A first embodiment of the invention is shown in FIG. 1. The functional part of the device is the microtube 2, typically a section of stainless steel or titanium hypodermic tubing (typically 100 to 300-micron internal diameter and having a typical working length from 25 mm to 100 mm) which is inserted into the tissue site of interest. Optical interrogation (via an optical fiber 3) of tissue or interstitial fluid proximal to the microtube tip 4 may be used to characterize the present tissue or fluid in real time, to detect transitions of tissue or fluid type as the microprobe insertion proceeds, or as real-time feedback information to control depth of insertion to a desired site. The optical interrogation may be done directly from the tissue or fluid by any of the well-known spectroscopy technologies in an optical spectroscopy system 5, or it may be done via a chemical sensor coating 6 at the tip of the optical fiber (such as a Ruthenium Dioxide coating whose fluorescence properties are responsive to Oxygen concentration). The optical fiber tip 6 may also be coated with an immobilized optical reporter material which reacts to a target analyte (neurotransmitter or other protein) molecule; this reaction may occur either directly to the target analyte or indirectly to a binding agent specific to the target analyte. A pump system 7 may delivered or extract fluid from the tissue site proximal to the aperture (perforation) array 8 via a fluidic port tube 9 and the annular cross-section internal clearance volume external to the optical fiber 3 and internal to the interior wall of the microtube 2. Intracranial pressure may be monitored by the pressure transducer 10 while the pump system 7 is not activated; with the pump system 7 activated the pressure transducer reads delivery pressure or sample pressure. A conductive lead 11 may allow an electrical signal to be sampled by an electrical sensing and stimulation system 12; it also enables the delivery of electrical energy to tissue in contact with the the microtube 2. Depth of the electrical interface to the tissue at the microtube 2 insertion site may be selectively controlled with an optional insulating sleeve 13 or coating on the exterior of the microtube 2. The internal volume of the microtube 2 is sealed at the microtube tip 4 by an impermeable seal 14.

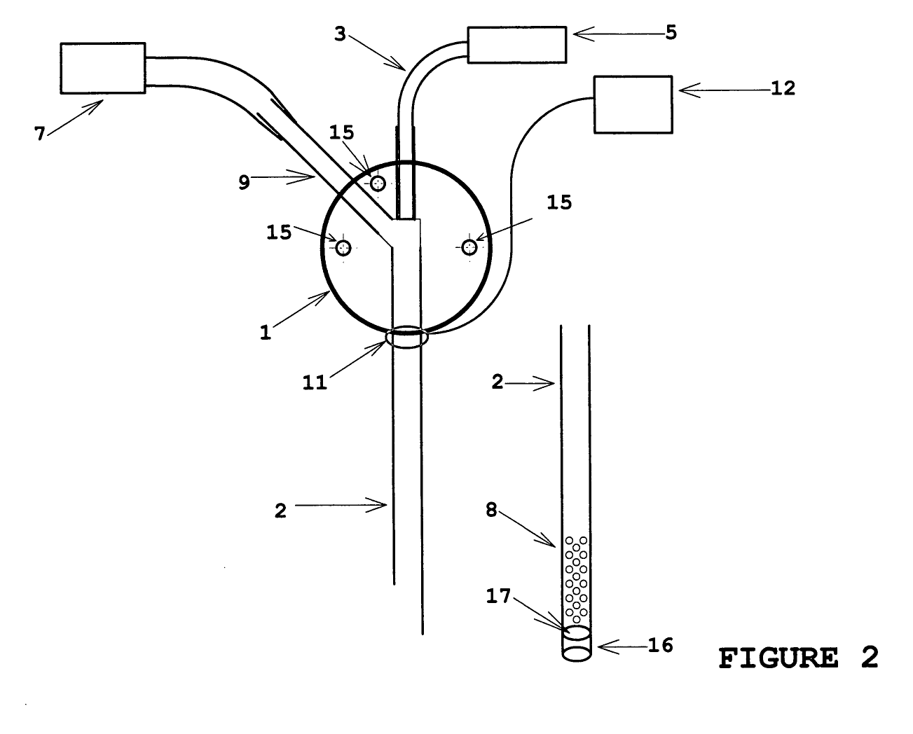

A second embodiment of the invention is illustrated in FIG. 2. A standard beveled hypodermic needle tip is shown; in addition to the features referenced as in FIG. 1, it also shows three mounting through-holes 15 which facilitate mounting the device to the experimental subject or to a separate stereotactic apparatus by means of pins, screws or adhesive. In this embodiment, the optical fiber 3 is mounted to extend only to the center of the multiport manifold body 1, so that the internal passage of fluid in the interior volume of the microtube 2 is unobstructed. This allows fluid delivery or sample extraction with minimum resistance, and thereby reduces fluidic phase lag for sampling or delivery. A variation of this embodiment is also shown in partial illustration, in which the open beveled end of the microtube 2 is replaced by an impermeable tip seal 16, and an aperture array of perforations 8 is located in the sidewall of the microtube 2. This configuration allows the interior bore of the microtube 2 to function as a long-path optical sample cell in which optical excitation is delivered by the end of the optical fiber 3 and may be reflected back by the inner surface 17 of the tip plug 16. Spectroscopic interrogation of the fluid in this cell is thereby enhanced. An additional possible function of this variation would be the optical interrogation of a chemical sensor coating or an enzyme-linked sensor coating on the inner surface 17 of the tip plug or on the end of the optical fiber 3. In this instance the aperture array 8 geometry would serve to suppress artifacts caused by large particles which could not pass through the perforations into the interior of the microtube 2. The dosed-end laser-perforated variation has the ability to selectively exclude (by size) suspended materials from extracted or delivered fluid; it also may minimize tissue damage at the sampling or delivery site by distributing the fluid volume interface over a larger area than the plain needle tip.

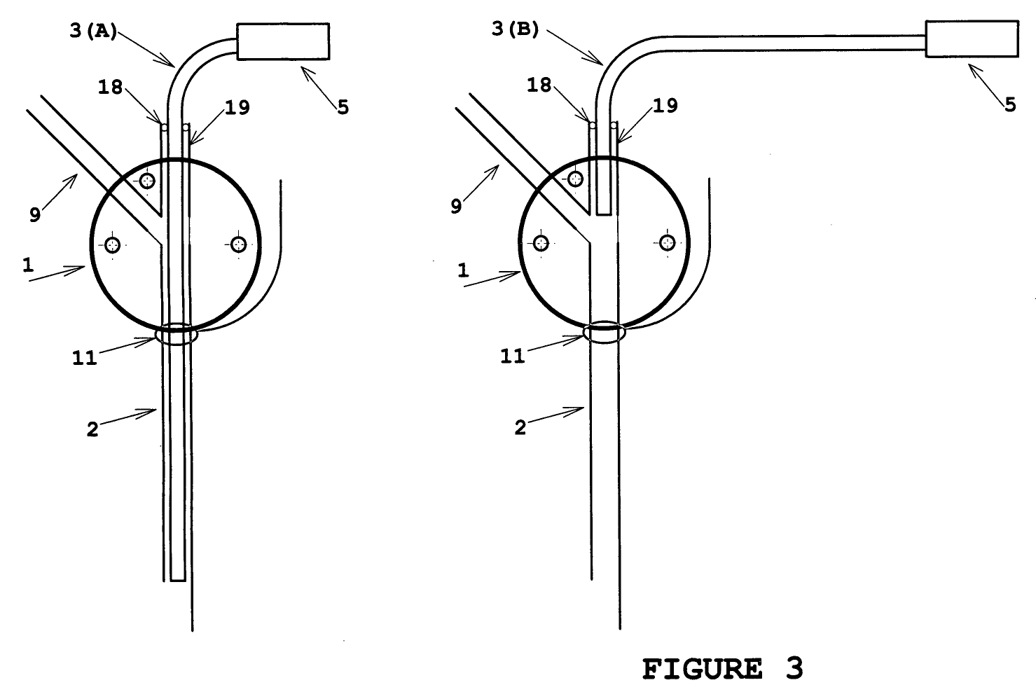

FIG. 3 presents a third embodiment of the invention, in which the optical fiber is held in an extensible/retractable mount (not shown). With the optical fiber 3(A) extended to its limit, the microprobe may be slowly inserted into a tissue sample of interest; optical interrogation by the optical spectroscopy system 5 of tissue or interstitial fluid proximal to the microtube tip 4 may be used to characterize the present tissue or fluid in real time, to detect transitions of tissue or fluid type as the microprobe insertion proceeds, or as a real-time feedback measure to control depth of insertion to a desired site. The optical interrogation may be done directly from the tissue or fluid by any of the well-known spectroscopy technologies, or it may be done via a chemical sensor coating at the tip of the optical fiber as described in the first embodiment (described above). The optical fiber 3(B) may then be withdrawn to its retraction limit and fluid may be extracted or delivered through the oblique port tube 9. The extension/retraction of the optical fiber 3(A) and 3(B) is enabled by passing the optical fiber 3 through a seal 18 which is retained in a fiber access port tube 19, which is fixed in the multiport manifold body 1.

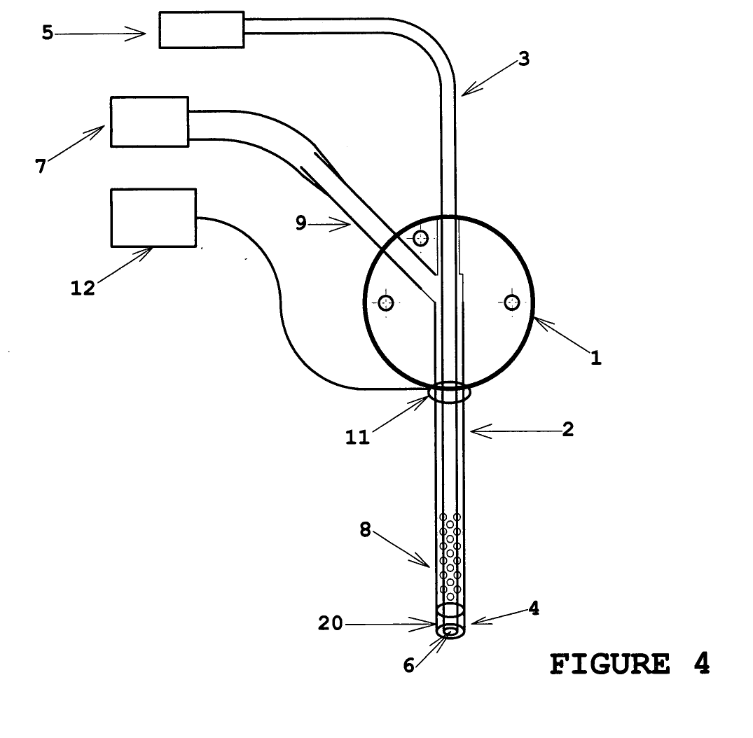

FIG. 4 presents a fourth embodiment of the invention, in which the optical fiber 3 extends through the tip 4 of the microtube 2 and is retained by an annular tip plug 20. Access to surrounding tissues for fluid sampling and delivery is via a linear or cylindrical array of laser-drilled apertures 8 located in the tubing wall near the microtube tip 4. An alternate implementation of this variation would be for fluid access through an annular porous plug, sieve, or honeycomb grid in place of the annular tip plug 20. As the microprobe is slowly inserted into a tissue sample of interest; optical interrogation of tissue or interstitial fluid proximal to the tip may be used to characterize the present tissue or fluid in real time, to detect transitions of tissue or fluid type as the microprobe insertion proceeds, or as a real-time feedback measure to control depth of insertion to a desired site. The information developed from the optical interrogation at the optical fiber end surface 6 may also be used to control the selection of one of several diagnostic or therapeutic agents or for real-time feedback control of volume or rate delivery of such an agent. The optical interrogation may be done directly from the tissue or fluid by any of the well-known spectroscopy technologies, or it may be done via a sensor coating at the optical fiber end surface 6 as described in the first embodiment above. A fluid sample may be extracted or delivered through the oblique port tube 9 as required. Potential applications of this configuration include administration of gene therapy or stem cell therapy agents, and performance characterization of diffusion-enhanced drug delivery systems for treatment of glioma or other lesions.

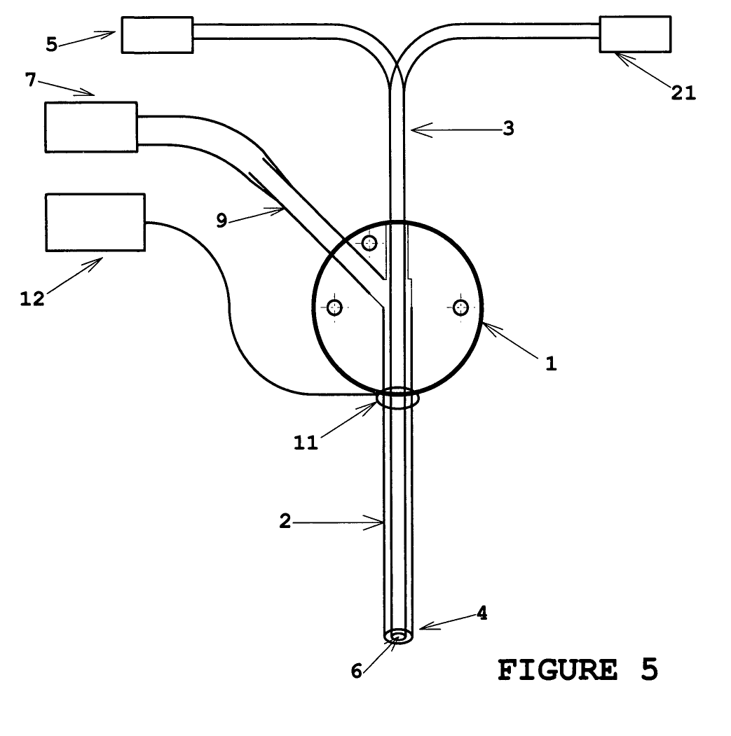

FIG. 5 depicts a fifth embodiment of the invention, which requires an optical power source 21 (for example, and ultraviolet or infra-red laser) separate from that used for optical interrogation, and a multiple-lumen optical fiber 3. For applications involving optically activated diagnostic or therapeutic agents, controlled illumination of desired wavelength, timing and intensity may be delivered to the tissue site by the optical fiber. This configuration may also be applied, for example, to stimulus-response experiments in which a nerve is optically stimulated by mid-range IR laser energy delivered via the optical fiber 3 to nerve tissue in the field of view of the optical fiber end surface 6, and the response is characterized by the simultaneous probe tip electrical signals carried over conductive lead 11 to a detector-amplifier in the electrical sensing and excitation system 12 and estimated oxygen uptake based on time-domain fluorescence measurements of ruthenium dioxide coatings on inert microspheres delivered to the site of interest via the fluidic port 9 from the fluidic pump system 7.

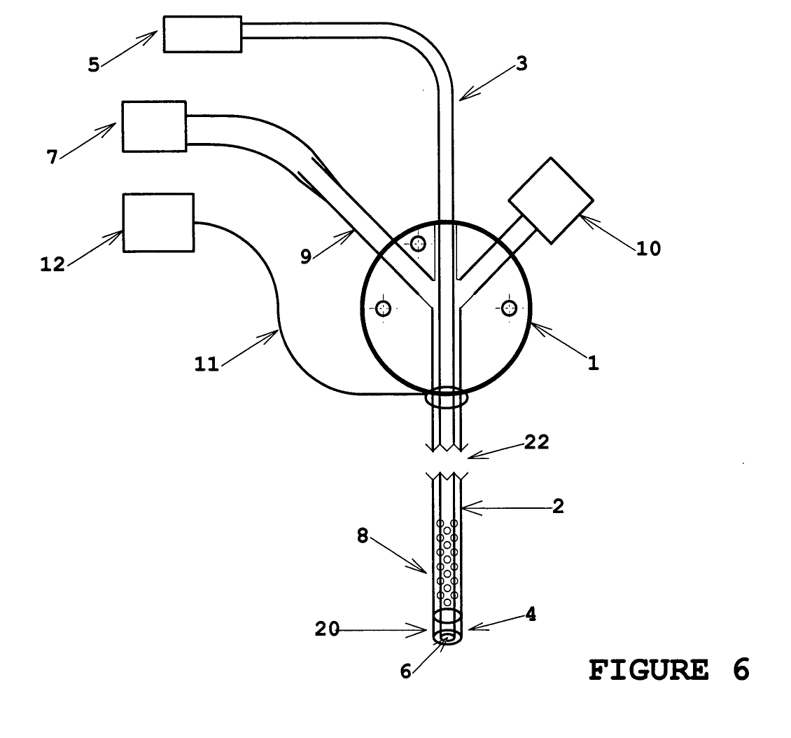

A sixth embodiment of the invention for “Clot-Busting” stroke applications is shown in FIG. 6. The main microtube 2 is constructed of flexible biocompatible tubing extended to a length of 1 to 2 meters. The extended microtube length is represented in FIG. 6 by the extension break 22. The flexible microtube is inserted into the affected blood vessel and advanced to the area of the clot. With tissue Plasminogen Activator (tPA) or other fibrinolytic agents supplied by a constant-flow pump in the fluidic delivery system 7, a delivery pressure signal may be monitored via pressure sensor 10 for (1) dangerously high resistance, or (2) sudden drop in resistance indicating lysis at the probe tip. Optical interrogation of material at the probe tip 4 for absorbance spectrum changes may reveal a restored flow of oxygenated blood through the clot area. Reversing the flow of the fluidic system may serve to extract tPA and debris-laden fluid from the site of the clot.

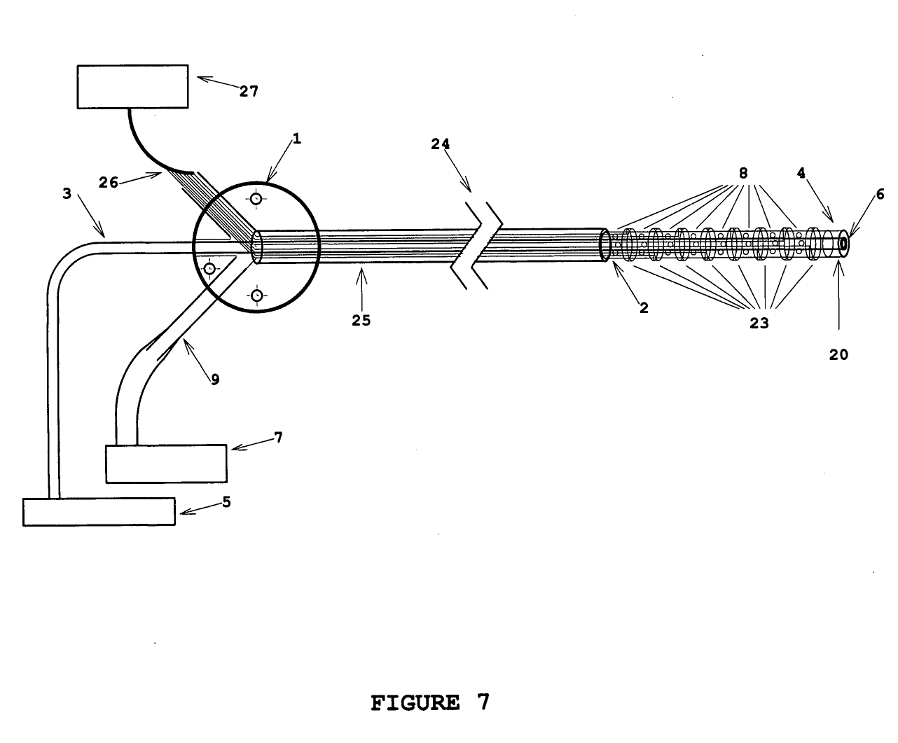

A seventh embodiment of the invention as presented in FIG. 7 may serve neuroscience research applications. This may be configured as (A) a “Depth Electrode Array” carrying five to eight ring electrodes 23 on the exterior surface of a microtube 2 (fabricated from a non-conductive material) having a length of about 320 mm. The ring electrodes are centered on a 5 mm to 10 mm pitch, at the tip 4 of the microtube 2. The seventh embodiment may also be configured as (B) a “Deep Brain Stimulation Electrode Array ” carrying 4 or more electrode rings 23 on the exterior of a microtube 2 of length about 375 mm, having the rings 23 centered on a 7.5 mm to 10 mm pitch.

Unlike prior versions of devices for these applications, this embodiment allows fluid sample extraction or delivery of fluid-borne reagents or drugs via laser-drilled perforations along the length of the array. It also allows optical spectroscopic interrogation of the fluid or tissue adjacent to the microprobe tip 4 via the optical fiber 3. An additional capability of this embodiment is the stimulation of nerve cells or other tissue adjacent to the probe tip or array. This may be done directly by delivery of 4 to 5 micron IR to the microprobe tip 4 via the internal optical fiber 3; it may also be accomplished indirectly by delivery of a fluid-borne reagent through the laser-drilled perforations 8, which is then activated by the optical energy at a suitable wavelength and intensity via the optical fiber. Stimulation may be tracked electrically from the electrode ring array 23 currents or voltages via a multiconductor cable 26 and a multiple-channel amplifier in the electrical sensing and stimulation system 2 7; information derived from those signals may be used to control fluid (drug) delivery via the fluidics port 9 or optical energy delivery via the optical fiber 3. Alternatively, electrical stimulation sourced by the conductive ring electrodes 23 may be controlled based on fiber-coupled optical spectroscopic measurements, which may or may not include a chemical or biologically reactive chromophore or fluorophore sensor coating on the optical fiber end 6. The extreme length (indicated by the “break” symbol 24) of the microtube 2 may require stiffening by an external sleeve 25.

This embodiment also may add an important decision-making and feedback capability for micro-probe localization (as in Deep Brain Stimulation procedures): stimulation and recording from different neuronal populations while advancing the micro-probe provides audio and graphical representation of the specific neuronal population.

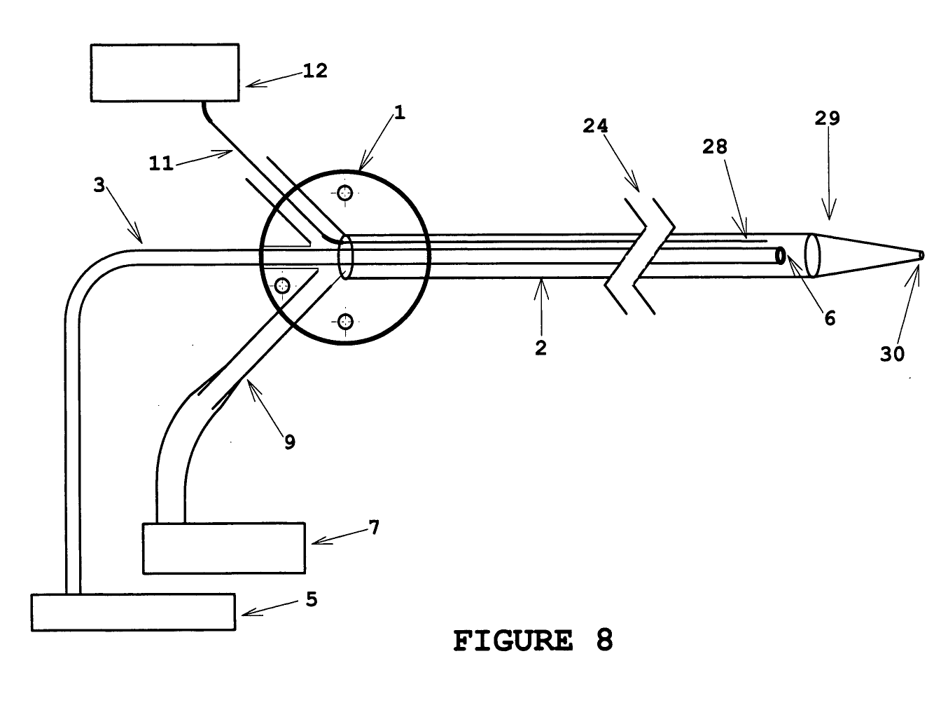

An eighth embodiment of the invention, configured as a “Patch Clamp Microprobe ” may serve an additional class of neuroscience research applications. FIG. 8 depicts a hollow microtube 2 constructed of a non-conductive rigid material such as glass, ceramic or polymer, which carries one or more optical fibers 3 and one or more conductive electrode wires 28 in its internal bore. The microtube 2 terminates in a hollow tapered tip 29, which ends in a 1-micron aperture 30. The microtube 2 may be many centimeters in length (as indicated with the “break” symbol 24).

In operation, the internal probe volume is filled with a suitable fluid via the fluidic port 9, then the microprobe is advanced to place the tip orifice 30 in contact with a target cell membrane. A gentle suction is then provided by the fluidic system 7, which creates a “gigaohm seal” between the probe and the membrane. Characteristics of the target cell (such as ion channel function) may then be observed by the usual patch clamp methods utilizing the wire electrode 28 signal via the signal lead 11. Additional measurements related to target cell membrane activity may be made simultaneously using optical spectroscopy methods via the optical fiber 3 in the microtube 2 interior. These measurements may also include use of fluorophore or chromophore-linked coatings on the optical fiber tip 6, or carried on the surface of particles suspended in the internal fluid; these coatings may selectively report particular analytes of interest resulting from cell membrane activity.

### Microprobe Functions

During research sample characterization processes or clinical diagnostic procedures one or more types of electrical, optical, fluidic, chemical or biological parameters may be measured or controlled. In such configurations of the system monitoring, testing, inducing and modulating of electrical, optical, fluidic, chemical or biological properties of the materials become possible in a real time. The main capabilities for measurement and control with this invention are:

Electrical Properties of the Sample Site Material.

The system is capable of measuring voltage potential or conductivity in dc and ac modes via single or multiple probes inserted in the area of interest. The system is capable of measuring and monitoring functional and structural response to electrical, mechanical, chemical, optical and biological stimuli.

Chemical/Biological Properties of the Sample Site Material.

System allows for extraction of different types of tissue and fluid from different insertion depth, separated one from another on the order of microns. Further characterization and diagnostics can be performed using available commercial technologies and methods. Electro-chemical responses may be measured in-situ using standard probe technology (example: pH or Calcium ion sensors) without extraction from the sample site.

Optical Properties of the Sample Site Material.

The material can be interrogated optically via use of absorbance, single and multiphoton fluorescence, Raman spectroscopy etc. These methods may be intensity or time domain, using UV, visible or IR wavelengths.

### Microprobe Application Parameters

The microprobe provides diagnostics, sampling, stimulation and fluid delivery, utilizing multiple modalities of physical, chemical, and biological parameters:

Optical Variable Parameters:

Wavelengths, intensities, period and duration of the optical pulse for mono and polychromatic excitation and emission.

Electrical Variable Parameters:

Wave form, frequency, pulse duration, voltage, current, amplitude.

Fluidic Variable Parameters:

Temperature, density, viscosity, flow rate, pressure, volume of the extracted or inserted material; diameters of the openings/apertures in the tool and different patterns of the aperture array.

Chemical Variable Parameters:

Composition, phase, pH, concentration, etc. of any chemical agent which could be modified or measured with available techniques.

Biological Variable Parameters:

Includes different types of biological agents (enzymes, lysing agents, antibodies, etc.) which can be inserted to the area of the interest through the openings in the tool.

### Microprobe Application Characteristics

Site-specific extraction of bodily fluids (for example: blood, CSF, interstitial fluid) in animals and humans.

Site-specific sampling of signals characteristic of bodily fluids or tissues with optical, electrical, mechanical or chemical sensors incorporated in or attached to the microprobe assembly.

Site-specific delivery of drugs or therapeutic agents in animals and humans.

Site-specific excitation or stimulation of bodily fluids or tissues by electrical, optical or other physical transducers incorporated in or attached to the microprobe assembly.

Site-specific sampling and delivery with real-time feedback control based on signals derived from optical, mechanical, chemical or electrical sensors and transducers incorporated in or attached to the microprobe.

1. (canceled)

2. (canceled)

3. A device providing multimodal access to small volumes of a biomaterial, comprising: a multiport micro-tube, manifold body having at least two intersecting bores linking the the micro-tube with ports, the micro-tube having a sealed tip and an aperture array near the tip and in the seal, the array serving for a first fluid infiltration inside the micro-tube from the biomaterial proximal to the micro-tube, wherein the size of all apertures A is selected to pass molecules having a size smaller than A.

4. The device according to claim 3, wherein the fluid extraction is provided by one or more pumps connected to a first port of the micro-tube, and wherein the pumps additionally may serve for a second fluid delivery to the biomaterial proximal to the micro-tube.

5. The device according to claim 3, wherein the pump is selected from a gravity-feed reservoir system, a peristaltic pump, a vacuum-driven and pneumatic-driven pump system.

6. The device according to claim 4, wherein the optical measurement of the biomaterial is provided through one or more optical fibers located inside the micro-tube, and wherein the optical fiber may optionally serve for the biomaterial optical excitation.

7. The device according to claim 3, wherein the aperture size is about 5 micron.

8. The device according to claim 6, in which the optical fiber may be retracted from the micro-tube to allow access to the site of interest by one or more of the other interrogation means.

9. The device according to claim 3, wherein the micro-tube internal diameter is 100 to 300-micron.

10. The device according to claim 3, wherein the first fluid pressure is sampled by a pressure transducer mounted integral to one of the ports on the micro-tube.

11. A device providing multimodal access to small volumes of a biomaterial at a site of interest, comprising: a micro-tube having access to the site of interest on one side and being connected to multiple treatment and measuring units via manifold body and multiple ports on the other side, the micro-tube allowing simultaneous and sequential treatment and monitoring of biomaterial characteristics.

12. The device according to claim 11, wherein the access to a first fluid at the site of interest is provided via a porous plug, screen, grid or membrane in the tip or sidewall of the micro-tube, the first fluid being a component of the biomaterial proximal to the micro-tube.

13. The device according to claim 11, wherein an optical energy source is used to illuminate via an optical fiber site of interest, the optical fiber collecting a returning light for diagnostic or therapeutic purposes.

14. The device according to claim 13, wherein an analytical sensor coating is applied to the end of the optical fiber proximal to the site of interest for the detection or measurement of physical, chemical or biological characteristics of that site.

15. The device according to claim 13, wherein a second fluid is delivered to the site of interest via the micro-tube for treatment or measurement of physical, chemical or biological characteristics of that site

16. The device according to claim 15, wherein for treatment of stroke, a fibrinolytic agent is delivered to a blood clot, and an action of the agent is monitored via a pressure and an optical spectroscopy measuring units, enabling delivery of additional agent under a feedback control protocol from the measuring units, wherein the monitoring is performed using an optical spectrometer attached to an optical fiber inserted in the micro-tube, the spectrometer receiving an optical signal with a spectrum indicating a level of oxygenation of blood passing the blood clot area; and wherein the pressure is measured by a pressure transducer mounted integral to one of the ports on the micro-tube.

17. A method of treatment and monitoring of various parameters at a site of interest in a biomaterial, comprising:
inserting a micro-tube to the site of interest sampling a first fluid around a tip of the micro-tube by filtrating a liquid component of the biomaterial through an aperture array located on sidewalls and tip of the micro-tube, the aperture size is selected to pass molecules with a diameter less than the aperture size;
monitoring at least one physical parameter of the first fluid using at least a first measuring unit connected to the micro-tube via at least one port of a manifold body attached to the micro-tube, and wherein the method may optionally include
delivering of a second fluid to the site of interest via the micro-tube.

18. The method according to claim 17, wherein the second fluid is a chemotherapeutic agent delivering into brain parenchyma, and the monitoring of the site during the delivering is performed by the measuring unit connected to the micro-tube

19. The method according to claim 18, wherein an information generated by the monitoring is used to control a volume of therapeutic agent delivery.

20. (canceled)

21. The method according to claim 17, wherein the aperture size is 5 micron.

22. (canceled)

23. The method according to claim 17, wherein the sampling and monitoring is performed during a seizure in vivo.

24. The method according to claim 17, wherein the micro-tube is fabricated using only materials compatible with standard Magnetic Resonance Imaging procedure in human or animal tissue.

25. (canceled)

26. The device method according to claim 17, wherein the site of interest is an outer membrane of a single cell neuron and the monitoring is performed in vivo.

27. (canceled)

28. (canceled)

29. (canceled)

30. (canceled)

31. The method according to claim 17, wherein the measuring units monitor the seizure prediction events.

32. (canceled)

33. The method according to claim 17, wherein an information to and from measuring units and a microcontroller is transmitted using a wireless or wired communications link.

34. The method according to claims 17, further comprising sampling the first fluid, monitoring the site of interest and optionally delivering the second fluid by at least one more micro-tube connected to the measuring units the additional micro-tube being inserted in the same biomaterial.

35. The method according to claim 17, wherein the apertures are produced by a process of laser micro-machining or photo-lithography.

36. (canceled)

37. The method according to claim 17, wherein the micro-tube is fabricated of Titanium tubing, and manifold body is fabricated of Acetyl.

38. The method according to claim 17, wherein the optical energy source is a fiber-coupled laser; the laser illuminating the site of interest via an optical fiber located inside the micro-tube; the optical fiber collecting a returning light from the site of interest; and monitoring by the measuring units includes processing of the returning light.

39.-42. (canceled)
